(19) United States
(12) Patent Application Publication
(10) Pub. No.: US 2011/0237600 A1
(43) Pub. Date: Sep. 29, 2011

(54) COMPOUNDS FOR IMPROVING LEARNING AND MEMORY

(75) Inventors: Karoly Nikolich, Emerald Hills, CA (US); Laszlo Nadasdi, Oakland, CA (US)

(73) Assignee: AMNESTIX, INC.

(21) Appl. No.: 12/991,839

(22) PCT Filed: May 11, 2009
(86) PCT No.: PCT/US09/43467

§ 371 Date: Jun. 3, 2011

Related U.S. Application Data
(60) Provisional application No. 61/052,600, filed on May 12, 2008.

(51) Int. Cl. A61K 31/496; C07D 401/12; A61P 25/00; A61P 25/18; A61P 25/22; A61P 25/24; A61P 25/28

(52) U.S. Cl. 514/253.05; 544/363

(57) ABSTRACT

The present invention provides a compound of Formula I: (I) and methods for improving memory in a subject by administering a therapeutically effective amount of the compound.

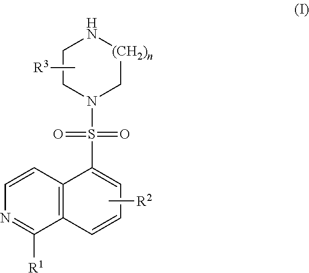

### INFORMATION ON RELATED APPLICATION

This application claims the priority benefit of U.S. Provisional Application No. 61/052,600 filed on May 12, 2008, which is hereby incorporated herein by reference.

### BACKGROUND

Human memory is a polygenic cognitive trait. Heritability estimates of ˜50% suggest that naturally occurring genetic variability has an important impact on this fundamental brain function. Recent candidate gene association studies have identified some genetic variations with significant impact on human memory capacity. However, the success of these studies depends upon preexisting information, which limits their potential to identify unrecognized genes and molecular pathways.

Recent advances in the development of high-density genotyping platforms have enabled the identification of some of the genes, particularly the KIBRA gene, responsible for episodic and long-term memory performance (Papassotiropoulos et al. Science 2006, 314, 475; WO 2007/120955). However, there is still no treatment available for subjects suffering from deteriorating episodic or long-term memory. Based on the identification of KIBRA as a central protein within the signaling pathway for stimulation of memory, it was found that administration of rho kinase 2 (ROCK) inhibitors, particularly Fasudil, can enhance learning and memory (Huentelman et al. Behavioral Neuroscience 2009, 123, 218; WO 2008/019395). In order to realize a treatment suitable for subjects suffering from deteriorating episodic or long-term memory, new compounds for the enhancement of learning and memory are needed.

### SUMMARY

In one aspect, compounds of the following Formula I are provided:

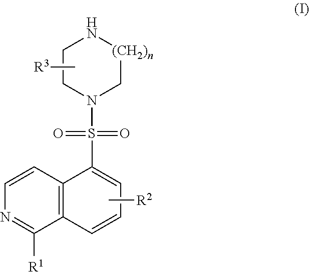

wherein R¹ is a member selected from the group consisting of hydrogen, C₁₋₆ alkyl, hydroxy, and halogen, preferably from the group consisting of hydrogen and C₁₋₆ alkyl; R² is C₃₋₈ cycloalkyl, whereas R² is localized at position 6, 7, or 8, preferably at position 8 of the isoquionline moiety; R³ is a member selected from the group consisting of hydrogen, and C₁₋₆ alkyl; and n is 0, 1, or 2, preferably 1 or 2; and salts, hydrates and solvates thereof.

In another aspect, methods are provided for improving learning and memory (including improving cognitive deficits in psychiatric disease such as schizophrenia, treating dementia, such as Alzheimer's disease, Pick's disease, Fronto-temporal dementia, vascular dementia, Kuru, Creutzfeld-Jakob disease, and dementia caused by AIDS/HIV infection), improving neural plasticity, and/or treating Alzheimer's disease in a subject, the method comprising administering to a patient in need thereof, a therapeutically effective amount of a compound of Formula I.

In another aspect, methods are provided for treating a patient for anxiety, depression, bipolar disorder, unipolar disorder or post-traumatic stress disorder, said methods comprising administering to said patient a therapeutically effective amount of a compound of Formula I.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1: Reaction scheme for the synthesis of 1-(1-chloro-8-cyclohexyl-5 isoquinoline-sulfonyl) 2-methyl-piperazine.

FIG. 2: Reaction scheme for the synthesis of 1-(8-cyclohexyl-5 isoquinoline-sulfonyl) 2-methyl-piperazine.

FIG. 3: List of kinases with the residual binding affinity to their active-site binding substrate in the presence of 1-(8-cyclohexyl-5 isoquinoline-sulfonyl) 2-methyl-piperazine measured in an AMBIT KinomeScan. The kinases are ranked according to their binding affinity to 11-(8-cyclohexyl-5 isoquinoline-sulfonyl) 2-methyl-piperazine.

FIG. 4 A: Induction of LTP by theta burst stimulation. Slopes (30 to 70% of maximum fEPSP amplitude) are plotted vs. time. LTP was induced after 15 min of control recording (arrow). The bars above data points indicate SEM.

FIG. 4 B: Effect of 1 μM 1-(8-cyclohexyl-5 isoquinoline-sulfonyl) 2-methyl-piperazine on LTP induction. Mean slopes (30 to 70% of maximum fEPSP amplitude) are plotted vs. time. LTP was induced after 30 min. of control recording (arrow) Black line indicates presence of 1-(8-cyclohexyl-5 isoquinoline-sulfonyl) 2-methyl-piperazine, bars indicate SEM, n=5 repeats.

FIG. 4 C: Effect of 10 μM 1-(8-cyclohexyl-5 isoquinoline-sulfonyl) 2-methyl-piperazine on LTP induction. Mean slopes (30 to 70% of maximum fEPSP amplitude) are plotted vs. time. LTP was induced after 30 min. of control recording (arrow) Black line indicates presence of 1-(8-cyclohexyl-5 isoquinoline-sulfonyl) 2-methyl-piperazine, bars indicate SEM, n=5 repeats.

FIG. 4 D: Effect of 100 μM 1-(8-cyclohexyl-5 isoquinoline-sulfonyl) 2-methyl-piperazine on LTP induction. Mean slopes (30 to 70% of maximum fEPSP amplitude) are plotted vs. time. LTP was induced after 30 min. of control recording (arrow) Black line indicates presence of 1-(8-cyclohexyl-5 isoquinoline-sulfonyl) 2-methyl-piperazine, bars indicate SEM, n=5 repeats.

### DETAILED DESCRIPTION

New compounds are provided that are useful for enhancing memory and learning, and for treating Alzheimer's disease. The compounds described herein can be used not only to treat memory loss, which is a symptom of Alzheimer's disease, but can be used to treat a cause of Alzheimer disease and delay onset or prevent development of the disease. In other aspects, the compounds can be used to treat anxiety, depression, bipolar disorder, unipolar disorder, and post-traumatic stress disorder.

Perhaps the two most studied proteins linked to memory are PKC and cyclic AMP response element binding protein (CREB). PKC family members play a purported role in memory due to their overexpression in several key brain regions, their involvement in memory processes across several species, their age-related alterations in activity in humans correlated with spatial learning deficits, and finally the evidence that PKC inhibition impairs learning and memory (Micheau, J. & Riedel, G. Cell Mol Life Sci 55, 534-48 (1999); Pascale, A., et al. Mol Neurobiol 16, 49-62 (1998); Sun, M. K. & Alkon, D. L. Curr Drug Targets CNS Neurol Disord 4, 541-52 (2005); Birnbaum, S. G. et al. Science 306, 882-4 (2004); Etcheberrigaray, R. et al. Proc Natl Acad Sci USA 101, 11141-6 (2004); Ruiz-Canada, C. et al. Neuron 42, 567-80 (2004)). Support for CREB as a memory-related gene include its defined role in long-term facilitation in the sea slug, Aplysia, and potentiation in rodents, the demonstration that the inducible disruption of CREB function blocks memory in mice, and exploration into compounds that alter CREB activity as memory enhancers (Josselyn, S. A. & Nguyen, P. V. Curr Drug Targets CNS Neurol Disord 4, 481-97 (2005); Carlezon, W. A., et al. Trends Neurosci 28, 436-45 (2005); Cooke, S. F. & Bliss, T. V. Curr Opin Investig Drugs 6, 25-34 (2005); Josselyn, S. A., Kida, S. & Silva, A. J. Neurobiol Learn Mem 82, 159-63 (2004); Martin, K. C. Neurobiol Learn Mem 78, 489-97 (2002); Lonze, B. E. & Ginty, D. D. Neuron 35, 605-23 (2002); Si, K., Lindquist, S. & Kandel, E. R Cell 115, 879-91 (2003); Chen, A. et al. Neuron 39, 655-69 (2003)). Additionally, there is mounting genetic evidence supporting the role of other proteins in memory including HTR2A, BDNF, and PKA (Alonso, M. et al. Learn Mem 12, 504-10 (2005); Bramham, C. R. & Messaoudi, E. Prog Neurobiol 76, 99-125 (2005); Papassotiropoulos, A. et al. Neuroreport 16, 839-42 (2005); de Quervain, D. J. et al. Nat Neurosci 6, 1141-2 (2003); Reynolds, C. A., et al. Neurobiol Aging 27, 150-4 (2006); Arnsten, A. F., et al. Trends Mol Med 11, 121-8 (2005); Quevedo, J. et al. Behav Brain Res 154, 339-43 (2004)).

KIBRA was recently identified in a yeast two hybrid screen as the binding partner for the human isoform of dendrin, a putative modulator of synaptic plasticity (Kremerskothen, J. et al., Biochem. Biophys. Res. Commun. 300, 862 (2003)). A truncated form, which was expressed in the hippocampus, lacks the first 223 aa and contains a C2-like domain, a glutamic acid-rich stretch and a protein kinase C (PKC) ζ-interacting domain (de Quervain, D. J. et al., Nat. Neurosci. 6, 1141 (2003)). PKC-ζ is involved in memory formation and in the consolidation of long-term potentiation (Bookheimer, S. Y. et al., N. Engl. J. Med. 343, 450 (2000); Milner, B. Clin. Neurosurg. 19, 421 (1972)). The C2-like domain of KIBRA is similar to the C2 domain of synaptotagmin, which is believed to function as the main Ca²⁺ sensor in synaptic vesicle exocytosis (Freedman, M. L. et al., Nat. Genet. 36, 388 (2004); Schacter, D. L. & Tulving E. Memory systems (MIT Press, Cambridge, 1994)). The memory-associated KIBRA haplotype block and SNP described in WO 2008/019395 map within the truncated KIBRA, which contains both the C2-like and the PKC-ζ-interacting domains. Taken together, evidence suggests a role for KIBRA in normal human memory performance.

In addition, KIBRA has high expression in brain, modulates Ca²⁺, is a PKC substrate, and is a synaptic protein. Several other genetic findings have allowed the identification of RhoA/ROCK as a target in memory, and Fasudil as a modulator to enhance memory, learning and cognition (Huentelman et al. Behavioral Neuroscience 2009, 123, 218; WO 2008/019395). CLSTN2 has high expression in brain, regulates Ca²⁺, and is a synaptic protein. CAMTA1 has high expression in brain, modulates Ca²⁺, and is a transcription factor. SEMA5A has high expression in the developing brain, and is involved in axonal guidance. TNR has high expression in the brain, is involved in the ECM, and assists in synapse maintenance. Finally, NELL2 also has high expression in brain, assists in neuronal growth, and shows enhanced LTP but impaired HPF-mediated learning. In addition, in situ hybridization of every one of the genetic targets shows expression in the mouse hippocampus.

The significance of the RhoA/ROCK pathway in normal memory function as well as in Alzheimer's cognitive decline (and likely other amnestic disorders) cannot be understated. Many devastating disorders include memory loss as a primary clinical characteristic and in the case of these disorders the RhoA/ROCK pathway may play a role in their overall severity, progression, or pathology. Even minimal prolongation before memory loss onset would be beneficial to patients suffering from these disorders.

Active-site dependent competition binding assays can be performed with hundreds of known kinases in parallel (Fabian et al., Nat Biotechnol. 2005, 23, 329; Karaman et al., Nat Biotechnol. 2008, 26, 127) in order to determine how compounds bind to both intended and unintended kinases. Such methods allow the assessment of the specificity of a kinase inhibitor.

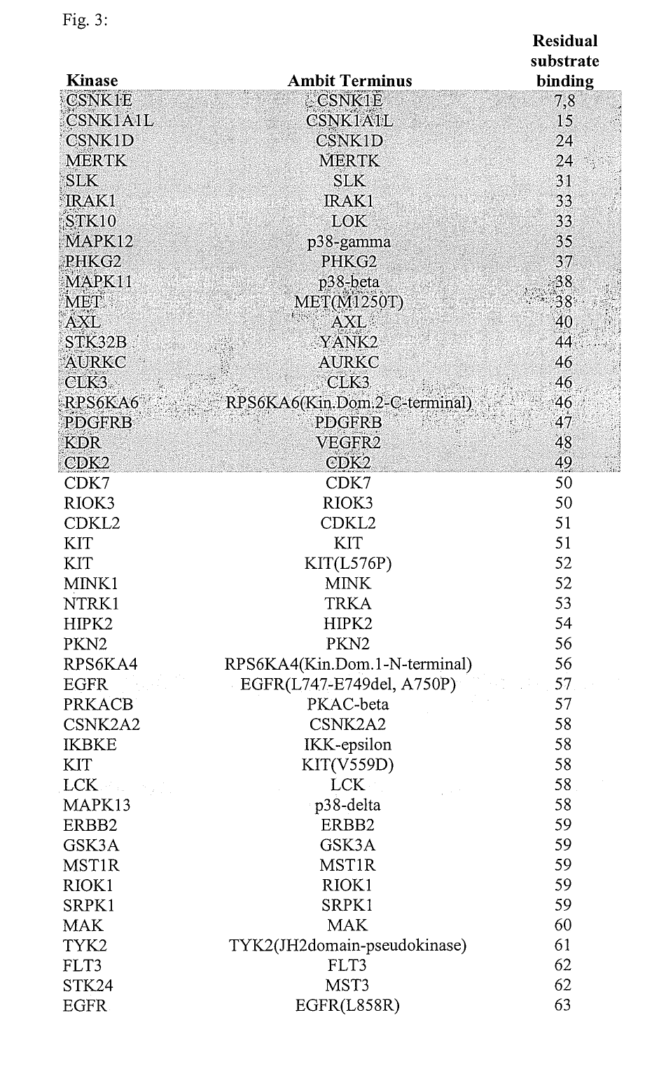

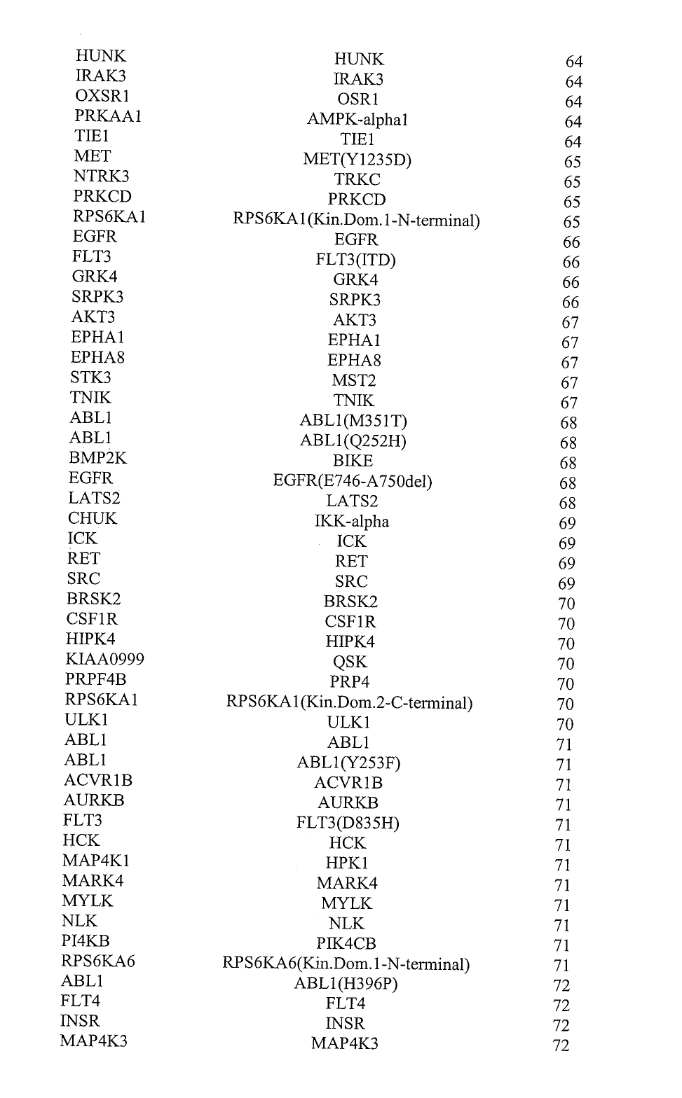

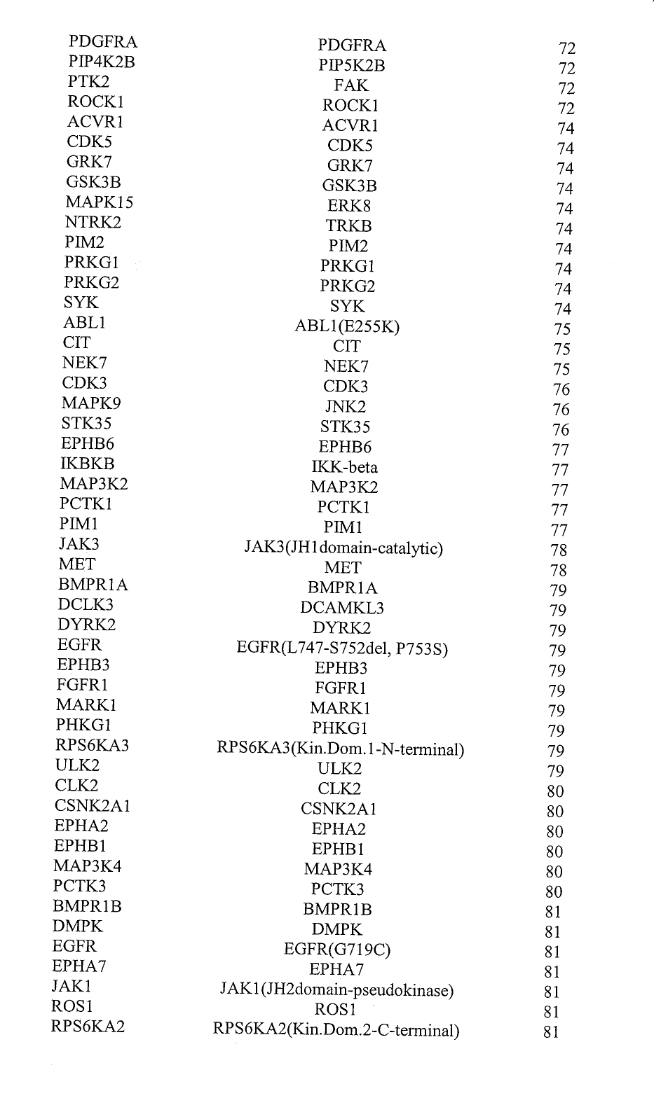

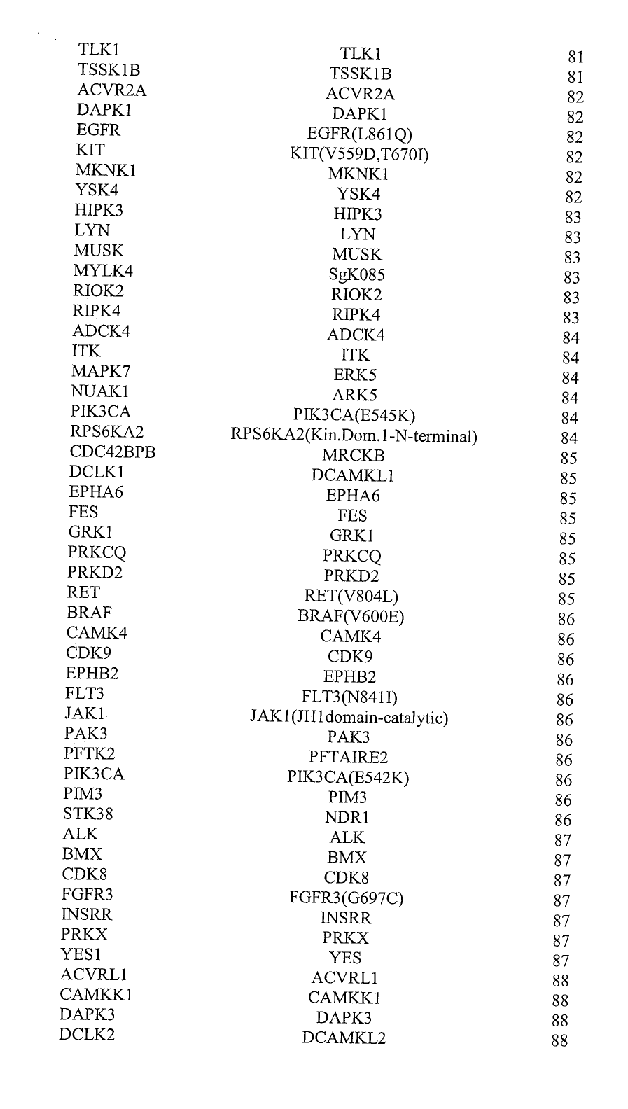

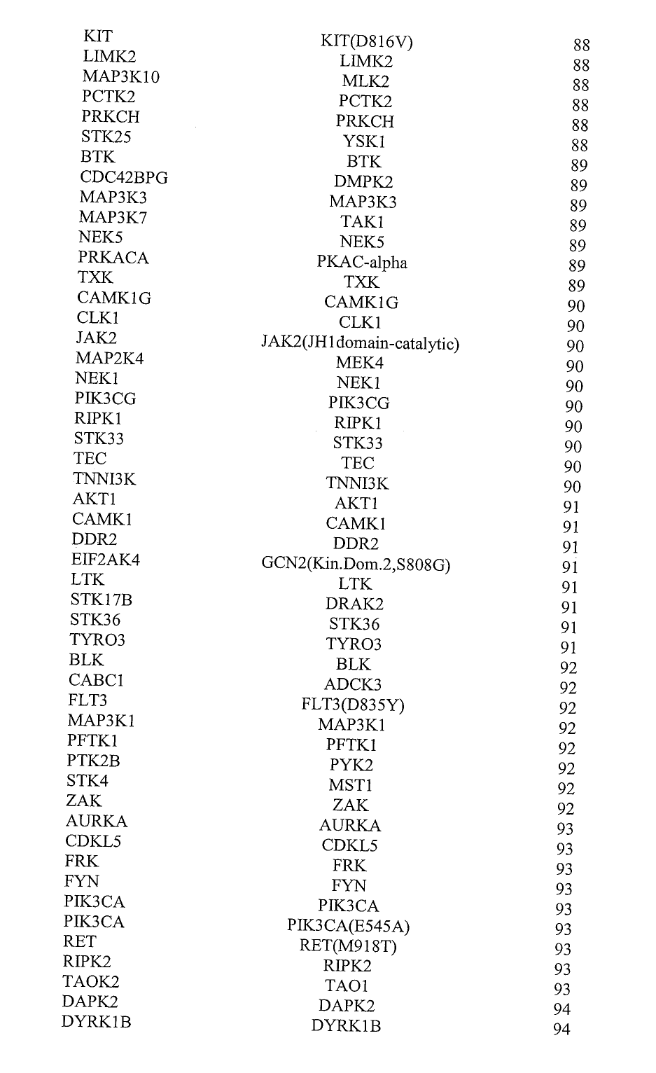

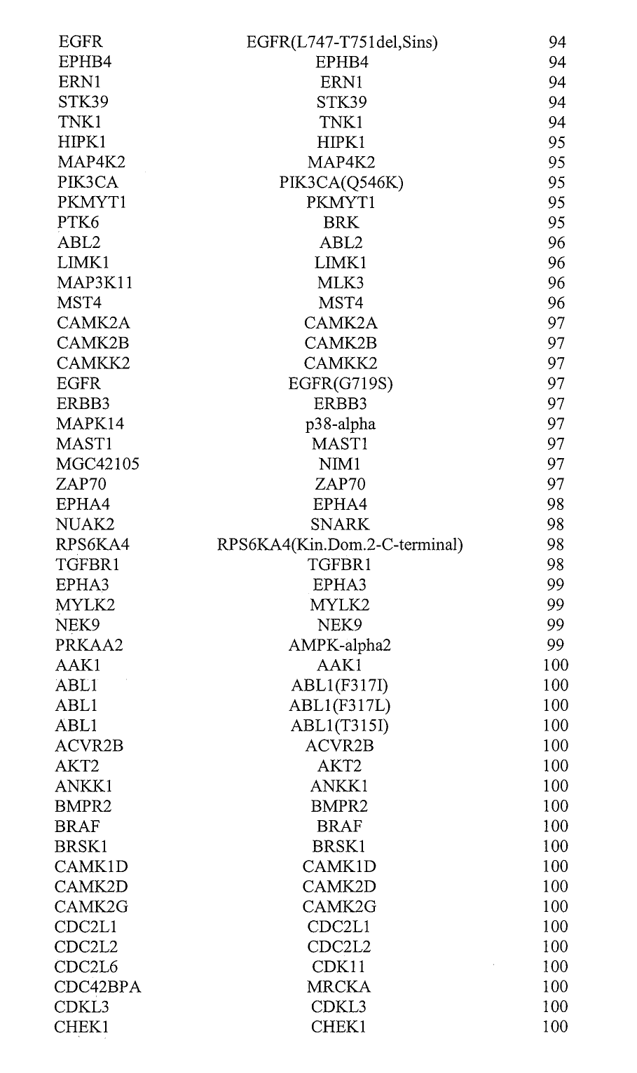

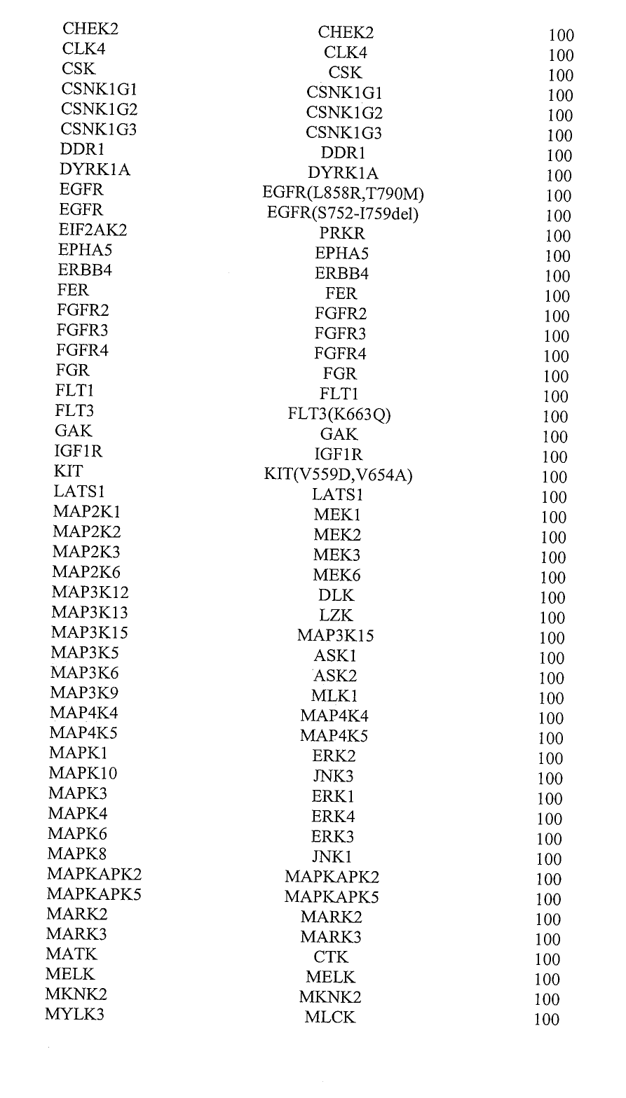

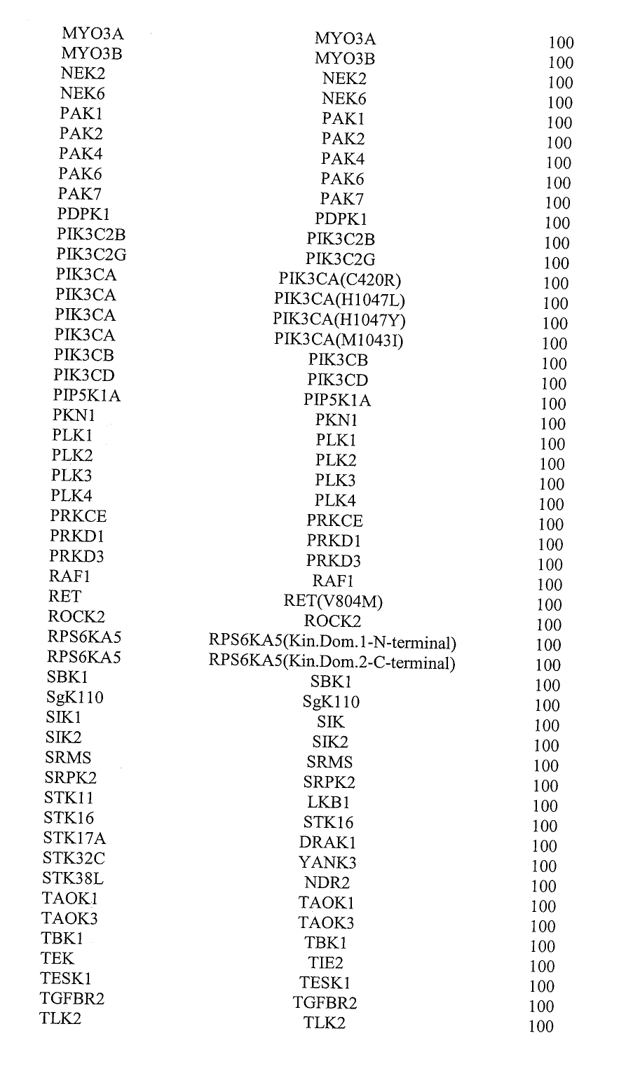

Compounds according to the invention show strong (more than 50%) binding within the active-site dependent competition binding assay only for relatively few kinases (e.g. CSNK1E, CSNK1A1L, CSNK1D, MERTK, SLK, IRAK1, STK10, MAPK12, PHKG2, MAPK11, MET, AXL, STK32B, AURKC, CLK3, RPS6KA6, PDGFRB, KDR, CDK2, See FIG. 3). These compounds are therefore useful as specific kinase inhibitors. They are suitable for treating conditions and diseases related to those kinases, namely CSNK1E, CSNK1A1L, CSNK1D, MERTK, SLK, IRAK1, STK10, MAPK12, PHKG2, MAPK11, MET, AXL, STK32B, AURKC, CLK3, RPS6KA6, PDGFRB, KDR, and CDK2.

To measure the effect of the administration of a compound on memory performance in vivo, various known animal tests can be used, e.g. the Sacktor-disc test which is a special form of active place avoidance with the experimental advantages of rapid hippocampus-dependent acquisition and persistent hippocampus-dependent recall (Pastalkova et al., Science 2006, 313, 1141). The apparatus consists of a slowly rotating platform, open to the room environment. The platform can be energized when the animal runs into a predefined sector. The rotation brings the animal into the shock zone, and the animal rapidly learns to avoid the shock by actively moving to the nonshock areas of the environment. Another possible in vivo memory test is the Morris water maze which was originally developed to test a rat's ability to learn, remember and to go to a place in space defined only by its position relative to distal extramaze cues (Morris et al., J Neurosci Methods 1984, 11, 47). Alternatively, one can use a radial arm maze to test an animal's memory. It consists of e.g. eight elevated arms around a octagonal shaped central platform. Animals can navigate through the maze using extramaze visual cues as orientation landmarks. Four of the arms are randomly baited with a small food pellet as reward and four are non-baited. Animals are allowed to explore the maze and memorize the locations of baited arms. In follow-up trials, running in a non baited arm is counted as a reference memory error, re-entry in the same arm is counted as a working memory error as well as re-entry of a previous visited baited arm. Advantageously, the radial arm maze can be used to test working memory as well as spatial memory simultaneously. Further known behavioral animal tests such as T-maze, open field, or object recognition can be used to assess animal memory by one skilled in the art. Such in vivo tests can be used on certain animal subpopulations such as aged animals, disease model animals, etc. in order to particularly assess the memory and memory enhancing effects within such a subpopulation. A form of classical conditioning is fear conditioning. It belongs to a model for studying emotional learning and memory. Conditioning means pairing of a conditioned stimulus e.g. a light or a tone with an unconditioned stimulus e.g. a mild shock. The unconditioned stimulus alone leads to a fear response. After several trials of repeated pairing the animal shows a fear response also to the conditioned stimulus alone. This is called a conditioned response. Pairing of different stimuli as described above is also known as cued fear conditioning, whereas contextual fear conditioning describes a fear response to the test chamber itself. The cued fear conditioning is sensitive to a brain structure called the amygdala, and the occurring contextual response seems to be more sensitive to the hippocampus. In animals both fear conditioning paradigms as well as active and passive avoidance paradigms can be used to demonstrate enhanced learning. Such in vivo tests can be used on certain animal subpopulations such as aged animals, disease model animals, etc. in order to particularly assess the memory and memory enhancing effects within such a subpopulation

The effect of long-term potentiation (LTP) which can be measured in vitro, is generally thought to correlate with memory. Stimulation of an afferent neuron or neuronal cell area results in membrane potentials of a downstream positioned neuron or neuronal cell area. Such membrane potentials are long-term potentiated at least over hours after stimulating the afferent neurons e.g. with a theta burst paradigm. Therefore, LTP is regarded as memory on cellular level. Electrophysiological LTP measurements on neurons incubated with a test compound in comparison to sham incubated neurons can be used to assess the compounds' potential to enhance memory (See generally Cooke and Bliss, Brain, 2006, 129 (1659), which is hereby incorporated by reference).

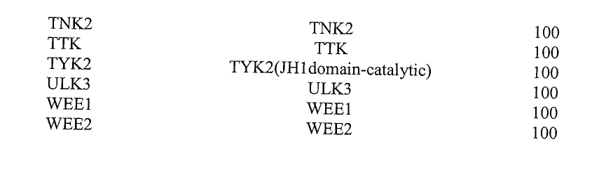

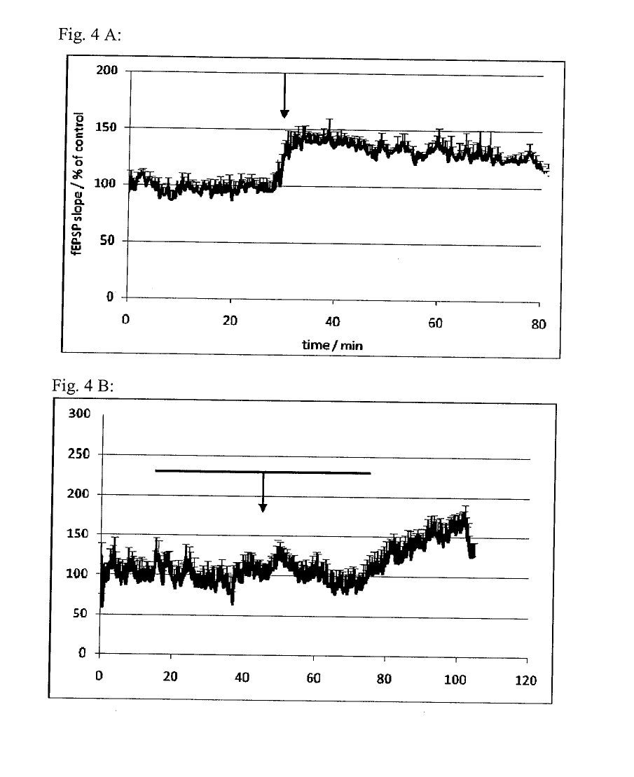

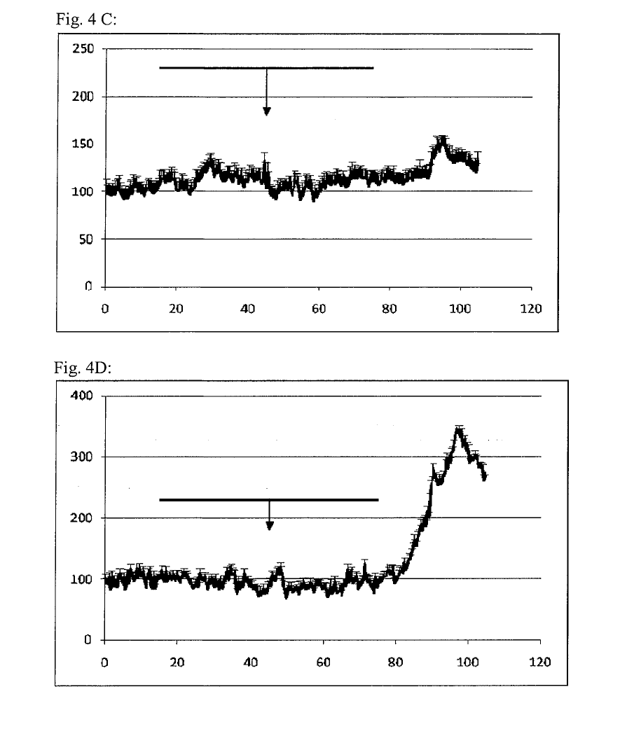

Using organotypic slice cultures of the rat hippocampus and washing in 1-(8-cyclohexyl-5 isoquinoline-sulfonyl) 2-methyl-piperazine at increasing concentrations, a complete block of LTP-induction is observed. In contrast, in control slices without 1-(8-cyclohexyl-5 isoquinoline-sulfonyl) 2-methyl-piperazine an LTP induction to about 140% of pre-stimulus levels is observed. When the compound is removed there is an increase in EPSP slopes, most obvious and very strong in the 100 μM concentration (See FIG. 4). This is to the inventor's knowledge a totally new observation. It has the appearance, that the LTP-inducing mechanisms of the cells are activated after the LTP-stimulus but that they are masked or blocked by the compound. Its removal releases this block and there is an overshooting response of the system. This initial masking of LTP could be, e.g. through the activation of chloride-channels that prevent the building-up of higher membrane potentials although NMDA- and AMPA-receptors are activated through the LTP-induction protocol. As soon as the Cl⁻-channels are inactivated the EPSP slope changes dramatically (“driving with the brakes on” and at some point release the brakes).

Thus, in addition to the effects on enhancing memory and cognition, the inventive compounds may be useful for a more complex modifying activity in cognition and memory formation. This might include the strengthening of only selected memories over others, e.g. reinforcing positive memories in contrast to negative memories, useful for example in post traumatic stress disorders due to traumatic experiences, extreme mourning and other triggers. Therefore, compounds of Formula I can be used for treating conditions and diseases like anxiety, depression, bipolar disorder, unipolar disorder, and post-traumatic stress disorder (PTSD).

There is general agreement that processes underlying memory-formation and learning include structural plasticity of neuronal networks and motility of dendrites or spines (for review see e.g. Tada & Sheng, Curr Opin Neurobiol., 2006, 16, 95). Neurite outgrowth is known to be influenced by Rho GTPases, a family of small GTPases with its members Rho, Rac and Cdc42. Rho GTPases are well known for their effects on the actin cytoskeleton and are therefore important regulators of cell motility and synaptic plasticity. Rho in its active GTP-bound form activates Rho kinase (ROCK), which subsequently activates myosin light chain, resulting in the rearrangement of the cytoskeleton and inhibition of axonal growth. It was observed that ROCK inhibitors like Fasudil increase neurite outgrowth in undifferentiated PC12 cells (Zhang et al., Cell Mol Biol Lett., 2006, 11, 12). In order to analyze the effect of a test compound with potential ROCK inhibition ability one can measure the neurite length of primary hippocampal neurons in cell culture in the presence of the test compound in comparison with a control assay without that compound. Alternatively to the increase in length it is also possible to determine the increase in complexity (Sholl analysis). Such compound which exhibit the ability to stimulate neurite outgrowth can be used for conditions in need of enhancement of cerebral plasticity and cognition.

Familial forms of Alzheimers Disease (AD) and Frontal Temporal Dementia (FTD) and the identification of the causative mutated genes have led to the generation of transgenic animal models for these diseases. The key player in AD is the amyloid precursor protein (APP). Mice overexpressing the mutant APP are the most widely used model to study memory impairment in AD (Ashe, Learn Mem. 2001, 8, 301; Chapman et al., Trends Genet. 2001, 17, 254; Goetz & Ittner, Nat Rev Neurosci. 2008, 9, 532). These mice carry different variants of the amyloid precursor protein (APP) and develop memory deficits over time as it is prominent from AD patients (e.g. animals with the so-called swedish mutation, Tg2576 (Hsiao et al., Science 1996, 274, 99)). These animal models can be utilized to test potential memory-enhancing compounds for their efficacy in an in vivo disease model.

Pathologies or neuropathologies that would benefit from therapeutic and diagnostic applications of this invention include, for example, the following:

diseases of central motor systems including degenerative conditions affecting the basal ganglia (Huntington's disease, Wilson's disease, striatonigral degeneration, corticobasal ganglionic degeneration), Tourette's syndrome, Parkinson's disease, progressive supranuclear palsy, progressive bulbar palsy, familial spastic paraplegia, spinomuscular atrophy, ALS and variants thereof, dentatorubral atrophy, olivo-pontocerebellar atrophy, paraneoplastic cerebellar degeneration, and dopamine toxicity;

diseases affecting sensory neurons such as Friedreich's ataxia, diabetes, peripheral neuropathy, retinal neuronal degeneration;

diseases of limbic and cortical systems such as cerebral amyloidosis, Pick's atrophy, Retts syndrome;

neurodegenerative pathologies involving multiple neuronal systems and/or brainstem including Alzheimer's disease, AIDS-related dementia, Leigh's disease, diffuse Lewy body disease, epilepsy, multiple system atrophy, Guillain-Barre syndrome, lysosomal storage disorders such as lipofuscinosis, late-degenerative stages of Down's syndrome, Alper's disease, vertigo as result of CNS degeneration;

pathologies associated with developmental retardation and learning impairments, and Down's syndrome, and oxidative stress induced neuronal death;

pathologies arising with aging and chronic alcohol or drug abuse including, for example, with alcoholism the degeneration of neurons in locus coeruleus, cerebellum, cholinergic basal forebrain; with aging degeneration of cerebellar neurons and cortical neurons leading to cognitive and motor impairments; and with chronic amphetamine abuse degeneration of basal ganglia neurons leading to motor impairments;

pathological changes resulting from focal trauma such as stroke, focal ischemia, vascular insufficiency, hypoxic-ischemic encephalopathy, hyperglycemia, hypoglycemia, closed head trauma, or direct trauma;

pathologies arising as a negative side-effect of therapeutic drugs and treatments (e.g., degeneration of cingulate and entorhinal cortex neurons in response to anticonvulsant doses of antagonists of the NMDA class of glutamate receptor, chemotherapy, antibiotics, etc.); and

learning disabilities such as ADD, ADHD, dyslexia, dysgraphia, dyscalcula, dyspraxia, and information processing disorders.

### I. DEFINITIONS

Memory systems can be classified broadly into four main types: episodic, semantic, working, and procedural (Hwang, D. Y. & Golby, A. J. Epilepsy Behav (2005); Yancey, S. W. & Phelps, E. A. J Clin Exp Neuropsychol 23, 32-48 (2001)). Episodic memory refers to a system that records and retrieves autobiographical information about experiences that occurred at a specific place and time. The semantic memory system stores general factual knowledge unrelated to place and time (e.g. the capital of Arizona). Working memory involves the temporary maintenance and usage of information while procedural memory is the action of learning skills that operate automatically and, typically, unconsciously. Episodic, semantic, and working memory are explicit (absolute) and declarative (explanatory) in nature while procedural memory can be either explicit or implicit, but is always nondeclarative (Tulving, E. Oxford University Press, New York, 1983); Budson, A. E., Price, B. H. Encyclopedia of Life Sciences (Macmillan, Nature Publishing Group, London, 2001); Budson, A. E. & Price, B. H. N Engl J Med 352, 692-9 (2005); Hwang, D. Y. & Golby, A. J Epilepsy Behav 8, 115-26 (2006)).

Normal aging states and disease states that impair memory include but are not limited to neurodegenerative disorders, head and brain trauma, genetic disorders, infectious disease, inflammatory disease, medication, drug and alcohol disorders, cancer, metabolic disorders, mental retardation, and learning and memory disorders, such as age related memory loss and age-associated memory impairment (AAMI), Alzheimer's disease, tauopathies, PTSD (post traumatic stress syndrome), mild cognitive impairment, ALS, Huntington's chorea, amnesia, B1 deficiency, schizophrenia, depression and bipolar disorder, stroke, hydrocephalus, subarachnoid hemorrhage, vascular insufficiency, brain tumor, epilepsy, Parkinson's disease, cerebral microangiopathy (Meyer, R. C., et al. Arm NY Acad Sci 854, 307-17 (1998); Barrett, A. M. Postgrad Med 117, 47-53 (2005); Petersen, R. C. J Intern Med 256, 183-94 (2004); Calkins, M. E., et al. Am J Psychiatry 162, 1963-6 (2005)), pain medication, chemotherapy (“chemobrain”), oxygen deprivation, e.g, caused by a heart-lung machine, anesthesia, or near drowning, dementia (vascular, frontotemporal, Lewy-body, semantic, primary progressive aphasia, Pick's), progressive supranuclear palsy, corticobasal degeneration, Hashimoto encephalopathy, ADD, ADHD, dyslexia and other learning disabilities, Down syndrome, fragile X syndrome, Turner's syndrome, and fetal alcohol syndrome, for example. In addition to disease, progressive memory loss is a normal byproduct of the aging process.

The term mild cognitive impairment (MCI) is used to refer to a transitional zone between normal cognitive function and the development of clinically probable AD (Winblad, B. et al. J Intern Med 256, 240-6 (2004)). A variety of criteria have been utilized to define MCI, however they essentially have two major themes: (1) MCI refers to non-demented patients with some form of measurable cognitive defects and (2) these patients represent a clinical syndrome with a high risk of progressing to clinical dementia.

The phrase “improving learning and/or memory” refers to an improvement or enhancement of at least one parameter that indicates learning and memory. Improvement or enhancement is change of a parameter by at least 10%, optionally at least about 20%, at least about 30%, at least about 40%, at least about 50%, at least about 60%, at least about 70%, at least about 80%, at least about 90%, at least about 100%, at least about 150%, at least about 200%, etc. The improvement of learning and memory can be measured by any methods known in the art. For example, compounds described herein that improve learning and memory can be screened using Morris water maze (See, e.g., materials and methods section). See, also, Gozes et al., Proc. Natl. Acad. Sci. USA 93:427-432 (1996). Memory and learning can also be screened using any of the methods described herein or other methods that are well known to those of skill in the art, e.g., the Randt Memory Test, the Wechler Memory Scale, the Forward Digit Span test, or the California Verbal Learning Test.

The term “spatial learning” refers to learning about one's environment and requires knowledge of what objects are where. It also relates to learning about and using information about relationships between multiple cues in environment. Spatial learning in animals can be tested by allowing animals to learn locations of rewards and to use spatial cues for remembering the locations. For example, spatial learning can be tested using a radial arm maze (i.e., learning which arm has food) a Morris water maze (i.e., learning where the platform is). To perform these tasks, animals use cues from test room (positions of objects, odors, etc.). In human, spatial learning also can be tested. For example, a subject can be asked to draw a picture, and then the picture is taken away. The subject is then asked to draw the same picture from memory. The latter picture drawn by the subject reflects a degree of spatial learning in the subject.

Learning disabilities is a general term that refers to a heterogeneous group of disorders manifested by significant difficulties in the acquisition and use of listening, speaking, reading, writing, reasoning, or mathematical abilities. Learning disabilities include ADD, ADHD, dyslexia, dysgraphia, dyscalcula, dyspraxia, and information processing disorders.

As used herein, “administering” refers to oral administration, administration as a suppository, topical contact, parenteral, intravenous, intraperitoneal, intramuscular, intralesional, oral, intranasal or subcutaneous administration, intrathecal administration, or the implantation of a slow-release device e.g., a mini-osmotic pump, to the subject.

As used herein, the term “alkyl” refers to a straight or branched, saturated, aliphatic radical having the number of carbon atoms indicated. For example, C1-C6 alkyl includes, but is not limited to, methyl, ethyl, propyl, butyl, pentyl, hexyl, iso-propyl, iso-butyl, sec-butyl, tert-butyl, etc.

As used herein, the term “halogen” refers to fluorine, chlorine, bromine and iodine.

As used herein, the term “heterocycle” refers to a ring system having from 5 to 8 ring members and 2 nitrogen heteroatoms. For example, heterocycles useful in the present invention include, but are not limited to, pyrazolidine, imidazolidine, piperazine and homopiperazine. The heterocycles of the present invention are N-linked, meaning linked via one of the ring heteroatoms.

As used herein, the term “hydrate” refers to a compound that is complexed to at least one water molecule. The compounds of the present invention can be complexed with from 1 to 10 water molecules.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

As used herein, the term “subject” refers to animals such as mammals, including, but not limited to, primates (e.g., humans), cows, sheep, goats, horses, dogs, cats, rabbits, rats, mice and the like. Preferably, the subject is a human.

As used herein, the terms “therapeutically effective amount” or “therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (See, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

### II. METHODS OF USE

The present invention provides methods for improving memory and learning by the administration of a compound of Formula I or salts, hydrates and solvates thereof. As indicated in the examples below, the compounds are used to enhance memory, improve neural plasticity, and/or treat Alzheimer's disease. The compounds can be administered orally, parenterally, or nasally, for example. For long term administration, lower doses can be used. The compounds according to the invention can be used in combination with other drugs to treat disease states or improve learning and memory. Furthermore, the compounds can be used as specific and potent ROCK inhibitors. Therefore, they are suitable for the treatment of ROCK related diseases, e.g. vasospasms following subarachnoid hemorrhage.

### III. COMPOUNDS

The present invention provides compounds of Formula I:

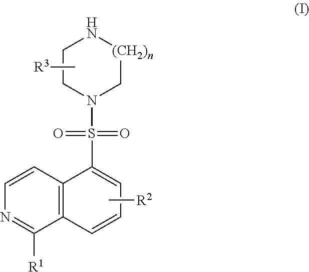

wherein R¹ is a member selected from the group consisting of hydrogen, C₁₋₆ alkyl, hydroxy, and halogen, such as from the group consisting of hydrogen and C₁₋₆ alkyl; R² is C₃₋₈ cycloalkyl, whereas R² is localized at position 6, 7, or 8, such as at position 8 of the isoquionline moiety; R³ is a member selected from the group consisting of hydrogen, and C₁₋₆ alkyl; and n is 0, 1, or 2, such as 1 or 2. In some embodiments were R¹ or R³ is an alkyl group, the group is a C₁₋₃ alkyl. The compounds of formula I can also be salts, hydrates and solvates thereof.

In general, compounds of Formula I, and their salts and hydrates, can be prepared using well-established methodologies and are based on the common knowledge of one skilled in the art. These are described, for instance, in U.S. Pat. Nos. 4,678,783 and 5,942,505 and European Patent No. 187,371, which are incorporated in their entireties herein by reference. More specific methodologies for representative compounds of the invention are presented in detail below.

In one embodiment, the compound is of the Formula:

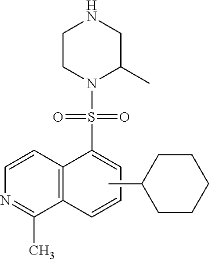

### or of the Formula:

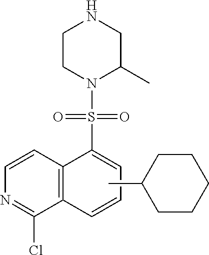

### or of the Formula:

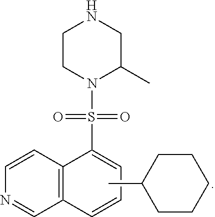

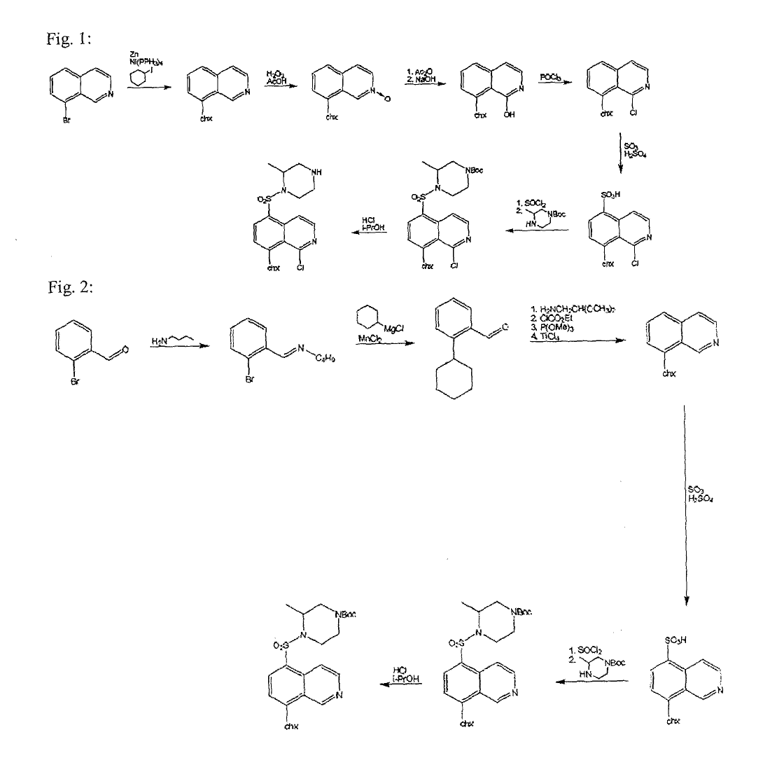

In other embodiments, the compound is 1-(1-chloro-8-cyclohexyl-5 isoquinoline-sulfonyl) 2-methyl-piperazine or1-(8-cyclohexyl-5 isoquinoline-sulfonyl) 2-methyl-piperazine. Illustrative syntheses of the compounds are depicted in FIGS. 1 and 2. Related compounds can be prepared analogously.

### IV. FORMULATIONS

The compounds of the present invention can be formulated in a variety of different manners known to one of skill in the art. Pharmaceutically acceptable carriers are determined in part by the particular composition being administered, as well as by the particular method used to administer the composition. Accordingly, there are a wide variety of suitable formulations of pharmaceutical compositions of the present invention (See, e.g., Remington's Pharmaceutical Sciences, 20^{th} ed., 2003, supra). Effective formulations include oral and nasal formulations, formulations for parenteral administration, and compositions formulated for extended release.

Formulations suitable for oral administration can consist of (a) liquid solutions, such as an effective amount of a compound of the present invention suspended in diluents, such as water, saline or PEG 400; (b) capsules, sachets, depots or tablets, each containing a predetermined amount of the active ingredient, as liquids, solids, granules or gelatin; (c) suspensions in an appropriate liquid; (d) suitable emulsions; and (e) patches. The pharmaceutical forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Preferred pharmaceutical preparations can deliver the compounds of the invention in a sustained release formulation.

Pharmaceutical preparations useful in the present invention also include extended-release formulations. In some embodiments, extended-release formulations useful in the present invention are described in U.S. Pat. No. 6,699,508, which can be prepared according to U.S. Pat. No. 7,125,567, both patents incorporated herein by reference.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

In practicing the methods of the present invention, the pharmaceutical compositions can be used alone, or in combination with other therapeutic or diagnostic agents.

### V. ADMINISTRATION

The compounds of the present invention can be administered as frequently as necessary, including hourly, daily, weekly or monthly. The compounds utilized in the pharmaceutical method of the invention are administered at the initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of disease diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, implanted micro pump or via a patch.

The pharmaceutical compositions can be administered to the patient in a variety of ways, including topically, parenterally, intravenously, intradermally, subcutaneously, intramuscularly, colonically, rectally or intraperitoneally. Preferably, the pharmaceutical compositions are administered parenterally, topically, intravenously, intramuscularly, subcutaneously, orally, or nasally, such as via inhalation.

In practicing the methods of the present invention, the pharmaceutical compositions can be used alone, or in combination with other therapeutic or diagnostic agents. The additional drugs used in the combination protocols of the present invention can be administered separately or one or more of the drugs used in the combination protocols can be administered together, such as in an admixture. Where one or more drugs are administered separately, the timing and schedule of administration of each drug can vary. The other therapeutic or diagnostic agents can be administered at the same time as the compounds of the present invention, separately or at different times.

### VI. EXAMPLES

### Example 1

### Preparation of 1-(1-chloro-8-cyclohexyl-5 isoquinoline-sulfonyl) 2-methyl-piperazine

1-(1-chloro-8-cyclohexyl-5 isoquinoline-sulfonyl) 2-methyl-piperazine is manufactured according to the synthesis scheme shown in FIG. 1.

### Example 2

### Preparation of 1-(8-cyclohexyl-5 isoquinoline-sulfonyl) 2-methyl-piperazine

1-(8-cyclohexyl-5 isoquinoline-sulfonyl) 2-methyl-piperazine was manufactured according FIG. 2. 112 g 2-bromobenzaldehyde and 61 ml n-butylamine dissolved in 350 ml toluene were heated for 3 h using a reflux condenser and subsequently stirred over night at ambient temperature. Solvent was distilled off resulting in 144 g N-(2-bromobenzyliden)butan-1-amine which is a red oil.

41 g of N-(2-bromobenzyliden)butan-1-amine were dissolved in 450 ml dry THF and 2.2 g manganese (II) chloride. After cooling to 0° C. 265 ml cyclohexyl magnesium bromide were added dropwise at 0-5° C. After stirring 1 h at 2° C., 150 ml saturated ammonium chloride solution was added dropwise at 2-7° C. Solution was extracted three times with ethyl ether. Combined organic phases were washed with 200 ml saturated sodium chloride solution and dried over sodium sulphate. Solvent was removed and residue chromatographically purified which yielded 26 g 2-cyclohexylbenzaldehyde which is a yellow oil.

26 g of 2-cyclohexylbenzaldehyde and 20.1 g 2-aminoacetylaldehyde dimethyl acetal were dissolved in 200 ml toluene and heated using a water separator. After removing the solvent the residue was dissolved in 100 ml dry THF. 18.2 ml ethyl chloroformate were added dropwise at −10° C. and solution was stirred for further 5 min. 22.6 ml trimethyl phosphite were added at ambient temperature and solution was stirred for further 16 h. After solvent was distilled off the residue was concentrated with toluene. The oily residue was dissolved under argon atmosphere in 450 ml dry dichloromethane and 126 ml titanium tetrachloride were added carefully. Solution was heated for 36 h using a reflux condenser and subsequently 1 L 20% sodium hydroxide solution was added. The solid matter was filtered and the aqueous phase of the filtrate was extracted two times with 200 ml dichloromethane and combined with the organic phase of the filtrate. The combined organic phase was extracted three times with 3 N hydrochloric acid. The combined aqueous phases were washed two times with 100 ml dichloromethane and shifted to alkaline pH with 10% sodium hydroxide solution. After extracting three times with 200 ml dichloromethane the combined organic phases were washed with water and saturated sodium chloride solution. After drying over sodium sulphate solvent was distilled off which yielded 4.5 g 8-cyclohexyl-isoquinoline, a yellow oil.

1 g of the 8-cyclohexyl-5-isoquinoline was dissolved in 5 ml ice cold sulphuric acid with subsequent dropwise adding of 5 ml oleum with further cooling. After stirring 2 h at 80° C. the solution was poured on ice water and the precipitate was filtered, washed with cold water, and vacuum dried which resulted in 1.2 g of 8-cyclohexyl-5-isoquinoline-sulfonic acid which is a brownish solid.

1.2 g of 8-cyclohexyl-5-isoquinoline-sulfonic acid was suspended in 15 ml thionyl chloride. After adding 0.1 ml DMF the solution was heated for 2 h using a reflux condenser. Solvent was removed under vacuum and oily residue was two times concentrated with dichloromethane. The foamy residue was suspended in 10 ml ice water and pH value was adjusted to pH 6-7 with saturated sodium bicarbonate solution. After extracting with 20 ml dichloromethane the organic phase was dried over magnesium sulphate and added dropwise to a solution of 4.95 g t-butyloxycarbony-3-methylpiperazine in 20 ml dichloromethane at 0° C. After 1 h stirring at 0° C. and 5 h at ambient temperature the solution was washed with 20 ml water, dried over magnesium sulphate, and concentrated. The residue was dissolved in 50 ml of 7 N hydrochloric acid in isopropanol and stirred for 2 h at ambient temperature. The solution was concentrated to dry residue which was dissolved in saturated sodium bicarbonate solution. The aqueous phase was extracted 3 times with dichloromethane and the combined organic phases were washed with 30 ml water and dried over magnesium sulphate. After removing the solvent the residue was chromatographically purified. This resulted in 470 mg of 1-(8-cyclohexyl-5 isoquinoline-sulfonyl) 2-methyl-piperazine as a colorless foam.

### Example 3

### Kinase Specificity Analysis

Based on a competition binding assay that quantitatively measures the ability of a test compound to compete with an immobilized, active-site directed ligand it is possible to scan the competitive effect of the test compound for a broad variety of kinases in parallel (KinomeScan, Ambit, San Diego, Calif., USA; Fabian et al., Nat Biotechnol. 2005, 23, 329). Based on this analysis it is possible to assess the inhibitory specificity of a test compound. The assay was performed with 1-(8-cyclohexyl-5 isoquinoline-sulfonyl) 2-methyl-piperazine in 10 μM concentration. As result of the assay one obtains the percentage of competition of the active-site directed ligand for each of the over 400 kinases of the test due to the incubation with the test compounds. FIG. 3 lists the kinases ranked according to their binding to 1-(8-cyclohexyl-5 isoquinoline-sulfonyl) 2-methyl-piperazine (compound A). Strong binding (more than 50%) of 1-(8-cyclohexyl-5 isoquinoline-sulfonyl) 2-methyl-piperazine is found only for the kinases CSNK1E, CSNK1A1L, CSNK1D, MERTK, SLK, IRAK1, STK10, MAPK12, PHKG2, MAPK11, MET, AXL, STK32B, AURKC, CLK3, RPS6KA6, PDGFRB, KDR, CDK2. Therefore, the compound is a very selective kinase inhibitor.

### Example 4

### In vitro LTP Analysis

LTP is thought to be suitable as in vitro model for the assessment of memory function. Therefore, it allows analysis of test compounds, e.g. the compounds of the invention, e.g. 1-(8-cyclohexyl-5 isoquinoline-sulfonyl) 2-methyl-piperazine for memory enhancing potential. Experiments were done on hippocampal slices from 3-4 week-old Wistar rats. The rats were sacrificed by decapitation without prior anesthesia. Brains were quickly removed and soaked in ice cold artificial cerebrospinal fluid (ACSF) containing: NaCl (124 mM), KCl (5 mM), Na2HPO4 (1.2 mM), NaHCO3 (26 mM), CaCl2 (2 mM), MgSO4 (2 mM), and glucose (10 mM), that was continuously bubbled with carbogen (95% 02, 5% CO2). Slices were then cut at 400 μm thickness using a vibratome and incubated in ACSF at room temperature for at least 1 h before starting recordings. All compounds used were diluted in ACSF at the concentrations needed and were prepared fresh on the day of recording from 100 mM stock solutions. To assure proper solubility of the compounds, stock solutions were made with DMSO. For recording, slices were transferred to a 4-channel slice chamber (Synchroslice, Lohmann Research Equipment) that allows simultaneous recording of 4 brain slices. Each slice was placed in a separate submerged type slice chamber where it was continuously superfused with temperature controlled (34° C.) ACSF or ACSF at a rate of 2 ml/min. Under visual control by a camera system, a bipolar stimulation electrode (Rhoades) was placed in the Schaffer collaterals and a single biphasic electrical stimulus of a duration of 200 μs and an amplitude of 200 μA was applied at 0.05 Hz. A platinum/tungsten electrode was then lowered into the CA1 dendritic layer under visual control until stable amplitudes of the recorded fEPSP were achieved. After a recording period of at least 10 min, the input-output relationship between stimulus amplitude and fEPSP amplitude was achieved separately for each slice. For recording, the stimulus amplitudes were chosen individually for each slice so that the resulting fEPSP showed 50% of the maximum amplitude from the IO curve. To induce LTP, 10 theta bursts were applied. Each burst consisted of 4 biphasic stimuli of 200 ms duration and 600 μA amplitude at a 10 ms interstimulus interval. The interburst interval was 200 ms. Each recording cycle started with a 15 min period in which electrical stimuli were applied at 0.05 Hz to assure stability of the fEPSP amplitude. Then, the test compound was washed in for a period of 30 min during which stimulation was continued at 0.05 Hz and fEPSPs were continuously recorded. LTP induction by theta burst stimulation was started 30 min after wash in. Recording was continued after LTP induction for at least 60 min, 30 min after LTP induction, compounds were washed out. All slices recorded simultaneously were treated with the same time schedule. From the recorded data, the amplitudes of the evoked fEPSP were automatically calculated by the recording software (Synchroslice data acquisition and analysis, LRE) as the negative peak of the postsynaptic signal with respect to baseline and plotted online. All recorded signals were digitally stored for later offline analysis, in particular for fEPSP negative slope calculation. From the stored single sweeps, the slope was calculated between 30% and 70% of the maximum fEPSP amplitude. To allow comparison of data obtained from different slices, fEPSP slopes were normalized to the control value (100%). Effects induced by applied substances were tested for statistical significance using either the Student's t-test of the Mann-Whitney Rank Sum Test, significance was assumed if p<0.05. Measurements for each experimental condition were repeated six times. Results are given as means from n=5 slices and standard deviation (SD).

Using organotypic slice cultures of the rat hippocampus and washing in 1-(8-cyclohexyl-5 isoquinoline-sulfonyl) 2-methyl-piperazine at three increasing concentrations (1 μM, 10 μM and 100 μM, See FIG. 4 B-D) in comparison to sham incubated slices (FIG. 4 A), a complete block of LTP-induction is observed. In contrast, in control slices without 1-(8-cyclohexyl-5 isoquinoline-sulfonyl) 2-methyl-piperazine an LTP induction to about 140% of pre-stimulus levels is observed. When the compound is removed there is an increase in EPSP slopes, most obvious and very strong in the 100 μM concentration.

### Example 5

### In Vivo Memory Assessment

Rats are one of the standard test systems for preclinical evaluation of age related cognitive impairments. Continuous subcutaneous administration of test compounds via osmotic mini pumps guarantees a stable plasma concentration and is therefore best for chronic application. In order to have a paradigm that investigates age-related memory impairment, 17 month old rats can be used. Alternatively transgenic dementia-modelling animals (e.g. Alzheimers disease) can be used. Animals are assigned to groups according to their treatment, one group only receives vehicle as control. Group sizes between 15 and 20 animals provide a proper statistical power depending on the number of groups investigated. For comparison of two groups, t-test statistics is used, for comparison of more than 2 groups, ANOVA corrected for multiple testing is applied. P-values of 0.05 are regarded as statistically significant. Experiments are performed in a blinded manner, including computer-generated probe randomizations and probe labeling, blindness of all experimenters to treatment identities until the end of the experiment, and separation of data analysis from experiment conduction. Animals are allowed to acclimate 1 week before starting the tests. Special care is taken to allow adequate access to food and water during trial, as well as for light-dark periods. One day before starting the tests osmotic mini pumps containing the test compounds or vehicle are implanted. The compounds according to the invention can be tested for their memory enhancing ability in vivo by that assays. In the following particularly suitable in vivo assays are described in detail.

Radial arm maze: One day after surgery rats are habituated for 4 days in the radial arm maze. After the habituation phase animals are tested in the radial arm maze for 14 days using four randomly baited arms with a small food pellet and four non baited arms. Running in a non baited arm is counted as a reference memory error, re-entry in the same arm is counted as a working memory error as well as re-entry of a previous visited baited arm. The run is over when all baited arms were entered or the time limit of 480 s was reached.

Sacktor-disk: The test starts with a habituation trial, in which the animal is exposed to the apparatus for 10 min without shock. This is followed by successive training trials, in which the animal receives an electric shock every time the animal runs into the shock zone. Training consists of eight 10 min training trials, separated by 10 min rest intervals in their home cage. The animals are then tested 24 h later in a single probe trial. The probe trial measures the retention of long term stored spatial information by the increase in time between the placement of the animal into the apparatus and the initial entry into the shock zone. In addition, the retention of both short term and long term stored information is tested by the decrease in time spent in the shock zone (which is expressed rapidly after a single training session).

Morris Water maze (MWM): On day one the visible platform test is first performed. Extramaze cues are hidden by curtains and a platform is placed with a visible mark in the first quadrant of the MWM. The animal is placed at the opposite quadrant and swims until it finds the platform with a maximal time of 60s. If it reaches the platform it is removed from the water and allowed to rest in its cage 30s between each trial. Four trials are executed with the visible platform located in each of the 4 quadrants. This provides parameters about the sensorimotor and motivational features of the animals, the latency to reach the platform, the velocity and the distance moved to reach the platform. On day two the animal is trained. In the pool with extramaze cues visible, it is placed at one of 4 randomly ordered start positions near the wall. The animal is supposed to swim to the submerged platform in a fixed position. If it fails to find the platform within 60s, it is placed on the platform for 60s. If it finds the platform within 60s, it is allowed to stay there 60s. The start location changes after each trial. The animal is trained to find the hidden platform with at least four trials per day. The animal is trained over as many days as it takes to reach the platform within 15s. This provides parameters about the ability of learning and motor performance, escape latency, swim speed and swim distance. After the training sessions the probe trial is done. The platform is removed, the animal is placed into the pool at the opposite quadrant than the platform was formally located and the animal swims 60s and is removed from the pool. This provides parameters on the percentage of time in quadrants of the MWM, number of crossings of the supposed platform positions, swim time, swim path length, swimming parallel to the wall, number of wall contacts and swimming speed.

1. A compound of Formula I: 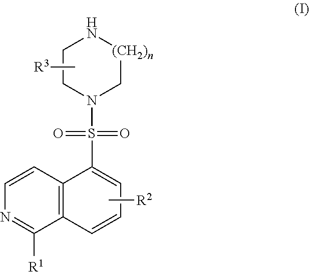
wherein
R1 is a member selected from the group consisting of hydrogen, C₁₋₆ alkyl, hydroxy, and halogen;
R² is C₃₋₈ cycloalkyl,
R³ is a member selected from the group consisting of hydrogen, and C₁₋₆ alkyl; and n is 0, 1, or 2.

2. The compound according to claim 1 that is 1-(1-chloro-8-cyclohexyl-5 isoquinoline-sulfonyl) 2-methyl-piperazine or 1-(8-cyclohexyl-5 isoquinoline-sulfonyl) 2-methyl-piperazine.

3. A method for improving memory in a subject, the method comprising administering to a patient in need thereof, a therapeutically effective amount of a compound according to claim 1.

4. A method for treating conditions related to a kinase selected of the group consisting of CSNK1E, CSNK1A1L, CSNK1D, MERTK, SLK, IRAK1, STK10, MAPK12, PHKG2, MAPK11, MET, AXL, STK32B, AURKC, CLK3, RPS6KA6, PDGFRB, KDR, CDK2 in a subject, the method comprising administering to a patient in need thereof, a therapeutically effective amount of a compound of claim 1.

5. The method of claim 4, wherein the conditions are selected from the group consisting of anxiety, depression, bipolar disorder, unipolar disorder, and post-traumatic stress disorder.

6. The method of claim 3, wherein the conditions are selected from the group consisting of Alzheimer's disease, schizophrenia, and mild cognitive impairment (MCI).

7. The method of claim 3, wherein the compound is 1-(1-chloro-8-cyclohexyl-5 isoquinoline-sulfonyl) 2-methyl-piperazine or 1-(8-cyclohexyl-5 isoquinolinesulfonyl) 2-methyl-piperazine.

8. The method of claim 4, wherein the compound is 1-(1-chloro-8-cyclohexyl-5 isoquinoline-sulfonyl) 2-methyl-piperazine or 1-(8-cyclohexyl-5 isoquinolinesulfonyl) 2-methyl-piperazine.

9. The method of claim 5, wherein the compound is 1-(1-chloro-8-cyclohexyl-5 isoquinoline-sulfonyl) 2-methyl-piperazine or 1-(8-cyclohexyl-5 isoquinolinesulfonyl) 2-methyl-piperazine.

10. The method of claim 6, wherein the compound is 1-(1-chloro-8-cyclohexyl-5 isoquinoline-sulfonyl) 2-methyl-piperazine or 1-(8-cyclohexyl-5 isoquinolinesulfonyl) 2-methyl-piperazine.
